(19) United States
(12) Patent Application Publication
(10) Pub. No.: US 2007/0148767 A1
(43) Pub. Date: Jun. 28, 2007

(54) Method of forming multicellular spheroids from the cultured cells

(76) Inventors: Mei-Ju Yang, Hsinchu City (TW); Wann-Hsin Chen, Hsinchu City (TW); Pei-Ju Lin, Hsinchu Hsien (TW); Chun-Hung Chen, Hsinchu (TW); Hsing-Wen Sung, Hsinchu (TW)

Correspondence Address: RABIN & BERDO, P.C.;Suite 500, 1101 14 Street, N.W., Washington, DC, 20005, US

(21) Appl. No.: 11/318,882

(22) Filed: Dec. 28, 2005

(51) Int. Cl. C12N 5/08; C12N 5/06

(52) U.S. Cl. 435/325; 435/366; 435/404; 435/370

(57) ABSTRACT

Provided herein are methods for producing multicellular spheroids or embryoid bodies suitable for providing cells in large scale for various medical applications. In one embodiment, a method of forming embryoid bodies is provided, which comprises culturing undifferentiated HES cells in a culture vessel pre-coated with cellulose and/or its derivatives. In another embodiment, a method of forming hepatic spheroids is provided, which comprises culturing hepatocytes in a culture vessel pre-coated with cellulose and/or its derivatives.

### FIELD OF THE INVENTION

The present invention relates to methods of producing multicellular spheroids or embryoid bodies that are suitable for providing cells for various medical applications such as cell therapy, drug screening and etc. More particularly, the present invention provides methods for the formation of multicellular spheroids or embryoid bodies by culturing cells in a culture vessel pre-coated with cellulose and/or its derivatives.

### BACKGROUND OF THE INVENTION

Three-dimensional (3-D) cell culture approach offers researchers a means to study cell growth, proliferation, and differentiation under conditions that emulate an in vivo environment and, to varying degrees, allow cell-cell and cell-extracellular matrix (ECM) interactions that might otherwise be severely constrained or precluded entirely in 2-dimensional (2-D) culturing condition (Edlman and Keefer, Experimental Neurology 2005, 192: 1-6).

Prior uses of the three-dimensional spheroid culture system are well established in tumor biology, where cells are cultured as multicellular tumor spheroids (MTS). Multicellular spheroid are used in studies such as tumor cell biology, therapy resistance, cell-cell interactions, invasion, drug penetration, modeling, tumor markers, nutrient gradients, tumor cell metabolism (Kunz-Schughart et al, 2004; Bates R C et al. Crit Rev Oncol Hematol. 2000, 36(2-3):61-74). Spheroid culture system has also been applied to various studies including mammary cell biology (Dontu and Wicha, Journal of Mammary Gland Biology and Neoplasia, 2005, 10:75-86); cytotrophoblast cells (Thomas K. Experimental Cell Research 2004, 297: 415-423); chondrocytes (Ursula Anderer and Jeanette Libera, J Bone Miner Res 2002; 17:1420-1429); hepatocyte (\check{C}ervenková et al, Biomed. Papers 2001,145: 57-60; Kazumori Funatsu, Artificial Organs, 2001, 25(3):194-200); bone marrow stromal cells (Braz. J. Med. Bio. Res. 2005, 38:1455-1462), neural stem/progenitor cells (Edlman and Keefer, Experimental Neurology 2005, 192: 1-6, Reynolds, 1992).

There are five most commonly approaches employed to produce 3-D cultures, which include: (1) organotypic explant cultures, in which whole organs or organ elements or slices are harvested and grown on a substrate in media; (2) stationary or rotating microcarrier cultures, in which dissociated cells aggregate around porous circular or cylindrical substrates with adhesive properties; (3) micromass cultures, in which cells are pelleted and suspended in media containing appropriate amounts of nutrients and differentiation factors; (4) free cells in a rotating vessel that adhere to one another and eventually form tissue or organ-like structures (so-called rotating wall vessels or microgravity bioreactors); and (5) gel-based techniques, in which cells are embedded in a substrate, such as agarose or matrigel, that may or may not contain a scaffolding of collagen or other organic or synthetic fiber which mimics the ECM (Edlman and Keefer, Experimental Neurology 2005, 192: 1-6).

In much work with cells, attachment of the cells to a surface is a must, and the surface is designed to give good attachment. However, in some circumstances, such as forming embryoid bodies from embryonic stem cells, attachment is a problem as it may cause unwanted differentiation of the cells. Most ES cells differentiation in vitro will go through a step called “Embryoid bodies formation”. ES cells are first aggregated to form embryoid bodies that contain 3 germ layers (including mesoderm, endoderm and ectoderm layers) and then are further induced into functional cells by either chemical stimulation (e.g., growth factors) , genetic engineering or mechanical stimulation. However, current cell therapy suffers a major drawback of unable to produce enough number of uniform phenotypic cells for wide applications such as embryo research, drug screening and cell transplant. Several approaches have been taken by the skilled artisans in this field in trying to produce large scale of ES cells derived functional cells through EBs formation methods for cell therapy, such approaches include the conventional hanging drop technique, low attachment method, encapsulated liquid suspension culture (ELSC), liquid bioreactor, semisolid culture by high viscosity medium and three-dimensional (3-D) culture, just to name a few.

The conventional hanging drop cultures typically consist of a defined number of cells allowed to aggregate in small fluid volume that hang form the tops of tissue culture dishes.

The low attachment method disclosed by Thomson et al. includes incubating primate ES cells under non-adherent conditions by either mechanically scraping or enzymatically dissociating the adherent ES colonies from the substrate or by agitating the incubation container (U.S. Pat. No. 6,602,711).

The encapsulated liquid suspension method is a process for generating embryoid bodies (EBs) in liquid suspension under high density by encapsulating EBs within a matrix, where individual cells or controlled aggregate of cells are encapsulated, and put inside a controlled stirred suspension bioreactor or in encapsulated liquid suspension culture (ELSC), where cells are prevented from aggregating and allowed to proliferate and differentiate into the desired cell type (WO 03/004626).

The semi-solid culture are cultures where cells are embedded in semi-solid media that prevents aggregation, for example, by preventing the collision of cells or cell aggregates due to its high viscosity (Stephen M. D. et al. Biotechnol. Bioeng. 2002 78(4), 442-453).

A 3-D culturing method was published by Rennard et al., which involves casting the EBs in a 3-D scaffolding material and a cell culture medium; and growing the EBs in the 3-D scaffolding material and cell culture medium. The 3-D scaffolding material is selected from albumin, collagen, gelatin, hyaluronic acid, starch, alginate, pectin, cellulose or cellulose derivatives, casein, dextran, polysaccharides or fibrinogen (US Application No.: 2005/0054100).

Although several approaches as described above have been proposed, yet each of them suffers at least one of the following drawbacks, such as time consuming, high manpower, complicating system, unable to scale up, cost inefficient, low spheroids formation rate, low differentiation rate and etc.

Accordingly, it is an object of the present invention to provide a simple, easy-to-use method with high spheroid formation rate for producing large amounts of spheroid forming cells as cell sources for medical applications in a relatively short period of time; such method is cost-effective and does not require high level of culturing techniques.

### SUMMARY

The present inventors have determined that cells cultured by the conventional hanging drop technique or by use of low attachment dishes (Petri dish) failed to generate spheroids in an efficient manner. As such, in one aspect, the present invention provides a method of forming spheroids or embryoid bodies, which is simple, cost-effective and with spheroid formation ratio over 90%, far superior than the results of the above-identified prior art methods. In another aspect, this invention provides spheroids of a differentiated cells or EBs of an undifferentiated stem cells that have been derived using the above method.

In one preferred embodiment, the invention provides a method of forming EBs by culturing the human embryonic stem (HES) cells in a culture vessel pre-coated with cellulose and/or its derivatives. In one preferred embodiment, methylcellulose (MC) was used as a coating substance of the culture vessel; however, other cellulose derivatives are equally applicable, preferably carboxymethylcellulose (CMC), hydroxymethylcellulose (HMC), hydroxyethylcellulose (HEC), hydroxypropylcellulose (HPC) and hydroxypropylmethylcellulose (HPMC).

In another preferred embodiment, the invention provides EBs that can be differentiated into a variety of cell lineages. In one preferred embodiment, EBs that are formed in accordance with the method of this invention are differentiated into hepatic-lineage cells or cells in three embryonic germ layers.

In another preferred embodiment, the invention provides a method of forming hepatic spheroids by culturing hepatocytes such as hepatoma cells (C3A cells) in a culture vessel pre-coated with cellulose and/or its derivatives. In still another preferred embodiment, hepatic spheroids are formed in accordance with the method of this invention.

These and other aspects and advantages will become apparent when the Description is read in conjunction with the accompanying Examples. It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a schematic drawing of the method of this invention used to form spheroids from the cultured cells;

FIG. 2 is the photograph showing the morphology of EBs formed on the surface of the MC-gel coated culture vessel in accordance with one preferred embodiment of this invention;

FIG. 3 illustrates the expression of three germ layer genes of EBs derived from HES cells grown on either PMEF feeders (lane 2) or feeder free system including mECM (lane 3) or hECM (lane 4) in accordance with one preferred embodiment of this invention; the expression of genes was detected by RT-PCR, undifferentiated HES cells grown on PMEF was used as a control (lane 1), and the reaction mixure without RNA was used as a negative control (lane 5);

FIG. 4 illustrates the immunohistochmical analysis of the expressed proteins originated from each of three germ layers of EBs (at day 30) derived from the HES cells maintain on PMEF feeder in accordance with one preferred embodiment of this invention; the normal isotype antibody was used as the negative control;

FIG. 5 illustrates the expression of hepatic genes of ES cells derived hepatic lineage cells analyzed by RT-PCR in accordance with one preferred embodiment of this invention;

FIG. 6A is the photograph showing the morphology of spheroids formed from the cultured hepatoma cells (C3A cells) on the surface of the cell culture dish for 4 days;

FIG. 6B is the photograph showing the morphology of spheroids formed from the cultured hepatoma cells (C3A cells) on the surface of the low attached petri dish for 4 days;

FIG. 6C is the photograph showing the morphology of spheroids formed from the cultured hepatoma cells (C3A cells) on the surface of the MC coated culture vessel in accordance with one preferred embodiment of this invention;

FIG. 7 illustrates the expression of hepatic-specific genes including α-FP, albumin, TAT, G6P and CYP 3A4 in C3A cells in attached cells (lane 1) or accordance with one preferred embodiment of this invention (lane 2);

FIG. 8A illustrates the measurement of a hepatic enzyme activity, cytochrome P450 3A4, by pentoxyresorufin (PROD) assay in attached hepatoma cells (C3A cells) on the surface of the cell cultured dished for 7 days; and

FIG. 8B illustrates the measurement of a hepatic enzyme activity, cytochrome P450, by pentoxyresorufin (PROD) assay in spheroid hepatoma cells (C3A cells) for 7 days in accordance with one preferred embodiment of this invention.

### DESCRIPTION OF THE INVENTION

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “pluripotent stem cell” refers to a cell that has the ability to self replicate for indefinite periods and can give rise to all adult cell types under the right conditions, particularly, the cell types that derived from all three embryonic germ layers—mesoderm, endoderm, and ectoderm.

The term “embryoid bodies or EBs” refers to three-dimensional (3-D) HES cell aggregates formed during culture that facilitate subsequent differentiation. When grown in suspension culture, ES cells form small aggregates of cells surrounded by an outer layer of visceral endoderm. Because their size, differentiation capacity, and gene expression profile resemble the early post-implantation embryo, these aggregates have been termed embryoid bodies or EB and are often employed as models of differentiation and gene expression in early development. ES cells grown and allowed to aggregate, they form small spheroid balls of cells call “simple embryoid bodies”. The simple embryoid bodies have an outer layer of large visceral and parietal endoderm cells. Upon further growth, they develop into cystic embryoid bodies with fluid-filled cavities and an inner layer of ectoderm-like cells.

Thus, the term “spheroids” refers to spherical-like 3-D aggregates of cells that formed during culture. In one preferred embodiment, hepatic spheroids or 3-D aggregates of hepatocytes are formed by the method of this invention.

The term “culture vessel” includes any vessel suitable for holding a liquid cell culture. Many culture vessels are well known in the art. Exemplary culture vessels include Erlenmeyer flasks, baffled flasks, dishes, plates, beakers, test tubes and etc.

All other acronyms and abbreviations have the corresponding meaning as published in journals related to the arts of chemistry and biology.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

All publications mentioned in this application are incorporated by reference to disclose and describe the methods and/or materials in connection with which the publications are cited. Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Methods, techniques, and/or protocols (collectively “methods”) that can be used in the practice of the invention are not limited to the particular examples of these procedures cited throughout the specification but embrace any procedure known in the art for the same purpose. Furthermore, although some methods may be described in a particular context in the specification, their use in the instant invention is not limited to that context.

The present invention is directed to an improved method for generating a large amount of EBs, ES cells derived functional cells or mature functional cells suitable for use in various clinical applications, such as cell therapy, drug screening and etc. by producing cultures of EBs suitable for further differentiation or spheroids of differentiated cells in large scale in a semi-solid environment.

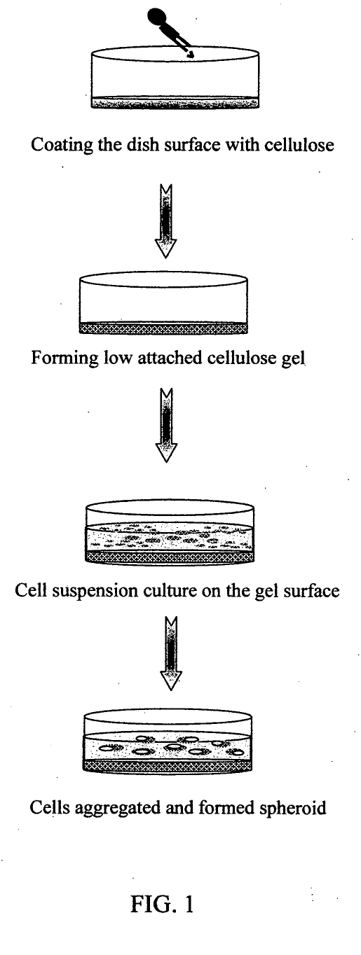

Referring to FIG. 1, which is a schematic diagram of the method for forming spheroids or embryoid bodies. Culture vessels are pre-coated with cellulose and/or its derivatives so as a thin film of low attached semi-solid gel is formed on the bottom surface of the vessel. Cells are then inoculated onto the semi-solid gel surface and remain in suspension to prevent physical aggregation of the cultured cells, so that more uniform, large diameter and viable EBs or spheroids are formed.

Thus in one embodiment, the invention provides a method of forming spheroids comprises culturing cells on a culture vessel pre-coated with cellulose and/or its derivatives.

The cellulose-coated culture vessels are prepared by pouring a small aliquot of cellulose solution onto the center of a culture dish at room temperature (˜20° C.), then the dish is tilted to evenly distribute the poured solution and forms a transparent thin film on the bottom surface of the dish. Then, the dish coated with a thin film of cellulose is incubated at an elevated temperature such as 37° C. for a pre-determined period of time such as an hour so that a gelled opaque layer, i.e., “Cellulose-gel”, is formed on the bottom surface of the culture dish. These cellulose-gel coated culture dishes are then used for formation of EBs or spheroids in subsequent experiments.

In one preferred embodiment, methylcellulose (MC) was used as a coating substance of the culture vessel; however, other cellulose derivatives are equally applicable, preferably carboxymethylcellulose (CMC), hydroxymethylcellulose (HMC), hydroxyethylcellulose (HEC), hydroxypropylcellulose (HPC) and hydroxypropylmethylcellulose (HPMC).

The suitable culture vessels for use in this invention include Erlenmeyer flasks, baffled flasks, dishes, plates, beakers, test tubes and etc. In a preferred example, cell culture dishes (Falcone®, Becton Dickinson Labware, Franklin Lakes, N.J., USA) were used.

In one preferred example, a method of forming embryoid bodies of HES cells is provided with ratio of cystic EBs formation over 90%, far superior than that of the prior methods including hanging drop technique and/or culture with low attachment dishes. The method of culturing HES on MEF feeder or feeder free culture system are described in details in another co-pending U.S. application Ser. No. 11/233,055 filed by the same applicant on Sep. 23, 2005, titled “Human Embryonic Stem Cells and Culturing Methods Thereof”, which is incorporated herein by reference. In brief, HES cells was cultured in a feeder-free system, which mainly composed of a substrate covered with extracellular medium (ECM) isolated from feeder cells pre-inactivated by gamma ray irradiation or by treatment with mitomycin C and a conditioned medium pre-conditioned by the feeder cells. The major ingredients of the conditioned medium are typically amino acids, vitamins, carbohydrates, inorganic ions, growth factors and some other bioactive substances. The inventors discovered that with the combinational use of ECM and a condition medium both prepared by the disclosed method, the undifferentiated growth of HES cells is significantly enhanced. The method of forming EBs of HES cell comprises culturing undifferentiated HES cells on a culture vessel pre-coated with cellulose and/or its derivatives. However, this method is equally applicable to any other cells that form spheroid-like structures. Such spheroid-forming cells may be of mammalian origin, murin origin and/or human origin, that include, but are not limited to, cardiomyocytes, hematopoietic cells, endothelial cells, neuronal cell, glial cells, kidney cells, hepatocytes, or vascular progenitor cells. In one preferred example, the hepatocytes cultured in accordance with the above-described method form spheroids (Example 2).

Applications of Cells Derived From The Method of This Invention

The present inventors have shown that by using semi-solid gel as a substrate for cell culture, three-dimensional spheroid-like structures such as embryoid bodies and hepatic spheroids are formed. The spheroids such as EBs obtained from the method of this invention can be used as a source to provide cells and tissues for transplantation. For instance, EBs can be grown in an environment to produce hematopoietic cells that can be used in bone marrow transplants or blood transfusions. In a preferred example, EBs formed by the method of this invention is differentiated into hepatic-lineage cells in the presence of growth factors such as bFGF (Example 1.3.2). This invention thus provides a way of generating cells suitable for subsequent medical purposes in large scale. Spheroid-forming cells such as HES cells and cells derived therefrom such as hepatocytes and etc. can be used in drug screen and the treatment of various disease include, but are not limited to, cancers, leukemias, autoimmune diseases, organ failure, tissue cloning and etc.

### EXAMPLES

The following Examples are provided to illustrate certain aspects of the present invention and to aid those of skilled in the art in practicing this invention. These Examples are in no way to be considered to limit the scope of the invention in any manner.

### Example 1

### Formation and Phenotypic Characterization of Embryoid bodies (EBs) of HES cells

1.1 Culturing of HES Cells

1.1.1 Preparation of Conditioned Medium

Conditioned medium for maintaining the culture of HES cell was prepared according to the following procedure. Briefly, primary mouse embryonic fibroblasts (PMEF) were plated in Dulbecco's Modified Eagle Medium (DMEM, obtained from Gibco Invitrogene) supplemented with 10% fetal bovine serum (FBS). Cell cultures were maintained at 37° C. and 5% CO₂ and in a water-saturated atmosphere until they reached confluence, then 10 μg/ml mitomycin C was added to inactivate the fibroblasts. The inactivated fibroblasts were then re-grown in DMEM medium supplemented with 20% FBS, 1 mM β-mercaptoethanl (obtained from Gibco Invitrogene), 1% non-essential amino acids (obtained from Gibco Invitrogene), 1% glutamine (obtained from Gibco Invitrogene), and 1% insulin- transferrin-selenium G supplement (ITS G supplement, obtained from Gibco Invitrogene) (See Richards, M. et al., Nat. Biotechnol., 20(9):933-936, 2002). FBS may also be omitted an/or substituted by serum replacement so as to obtain a serum-free growth medium. The medium in which the inactivated fibroblasts have been grown for at least 1 day was then collected for immediate use or stored at −80° C. for future usage. The medium thus collected was termed “conditioned medium”. Based on the requirements of the cultured cells, the growth medium may contain other ingredients without limited to those discussed herein.

1.1.2 Preparation of Extracellular Matrix (ECM)

Extracellular matrix for maintaining HES cells was prepared according to the following procedure. Briefly, primary mouse embryonic fibroblasts (PMEF) or human foreskin fibroblasts (HFF, obtained from Animal Technology Research Institute, Taiwan) were grown in DMEM medium supplemented with 10% FBS. When the cells reached 90% confluence, 10 μg/ml mitomycin C was then added to inactivate the fibroblasts. These inactivated cells were trypsinized, counted, re-plated in the culture dish, and confluence cultured for at least 2 days, then were lysed with 0.05N NaOH or 0.1% trinitrotoluene (obtained from Sigma) for a period of 1-15 min and rinsed with Phosphate Buffered Saline (pH 7.4) (1×, obtained from Gibco Invitrogene) to remove organelles and nucleus. The extracellular matrix of PMEF or HFF thus prepared can be used fresh or stored away for future use in PBS at 4° C. for at least 9 months.

1.1.3 Culturing of HES Cells

HES cells including HES-3 (ESI cell international) and TW1 (Industrial Technology Research Instituteand Lee Woman's Hosp., Taiwan) cell lines, were counted and plated onto culture dishes covered with feeder cells or the ECM of Example 1.1.2 and incubated with ES medium or the conditioned medium of Example 1.1.1. Culture medium was replaced every 1-2 days and cell cultures were maintained at 37° C. and 5% CO₂ and in a water-saturated atmosphere for 6-8 days. Undifferentiated cells were subcultured every 6-8 days using dispase (5 unit/ml, BD) and mechanically sliced into several pieces by use of pulled glass capillaries.

1.2 Formation of EBs

1.2.1 Preparation of Aqueous Methylcellulose Solutions

Aqueous methylcellulose (MC) solutions in various concentrations (9-12%, w/v) were prepared by dispensing weighted MC powders (M 7027, cell culture grade, Sigma, St. Louis, USA) in heated water with the addition of phosphate buffered saline (PBS) in various concentrations (0.25×˜1×) at 50° C. The osmolarities of the prepared MC solutions were then measured using an osmometer (Model 3300, Advanced Instruments, Inc., Norwood, Mass., USA).

1.2.2 Preparation of MC-Gel Coated Culture Vessels

The prepared MC solutions of Example 1.2.1, which had a gelling temperature below 37° C., were used to coat culture vessels such as cell culture dishes (Falcone®, Becton Dickinson Labware, Franklin Lakes, N.J., USA). First, 450 μl of a MC solution was poured onto the center of a culture dish at room temperature (˜20° C.), then the dish was tilted to evenly distribute the poured MC solution and formed a transparent thin film on the surface of the dish. Then, the MC-coated culture dish was incubated at 37° C. (or 50° C.) for one hour so that a gelled opaque layer, i.e., “MC-gel”, was formed on the surface of the culture dish. These MC-gel coated culture dishes were then used for formation of EBs or spheroids in subsequent experiments.

1.2.3 Formation of Embryoid bodies (EBs)

The HES pieces of Example 1.1.3 were transferred to the MC-gel coated culture dishes of Example 1.2.2 and remained suspended in a DMEM medium supplemented with 20% FBS, 1 mM β-mercaptoethanol (Gibco Invitrogene), 1% non-essential amino acids (Gibco Invitrogene), 1% glutamine (Gibco Invitrogene), and 1% insulin- transferrin-selenium G supplement (ITS G supplement, obtained from Gibco Invitrogene) for EBs formation, and the medium was changed every 2 to 3 days.

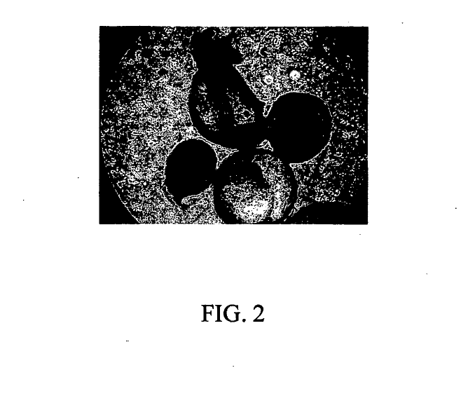

Following culture in suspension for 11 days, percentage of cystic EBs formation for HES cells cultured in MC-gel coated culture vessel was 95%, whereas the percentage of cystic EBs formation for HES cells that were cultured by the conventional hanging drop technique or by low attachment dish was 85% or 60%, respectively. The quantified results were summarized in Table 1, and the morphology of EBs formed on the surface of the MC-gel coated culture vessel of example 1.2.2 was illustrated in FIG. 2.

TABLE 1
The Quantified Results of the Cystic Ratio of EBs Formed by Various
Culturing Methods

| | Culturing Method | | |
|---|---|---|---|
| | | Low Attach- | MC-gel Coated |
| | Hanging Drop | ment Dishes | Dishes |
| Cystic Ratio of EBs | 85 | 60 | 95 |
| formed at 11 days (%) | | | |

1.3 Phenotypic Characterization of EBs of Example 1.2.3

1.3.1 Differentiation Capability of EBs of Example 1.2.3 Characterized by the Expression of Genes or Proteins of Cells of Each of the Three Germ Layers

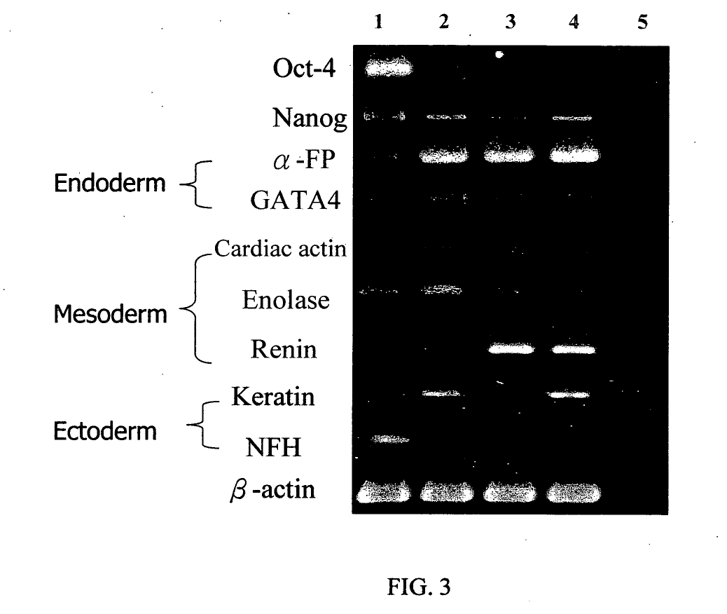

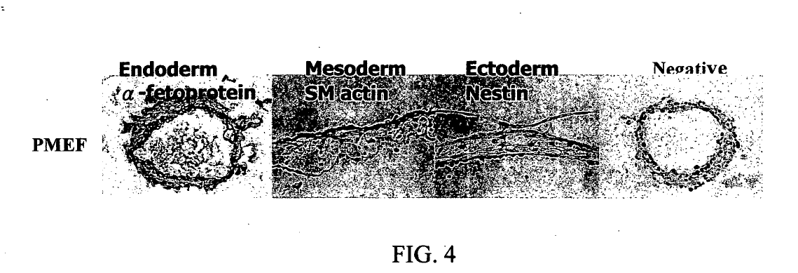

This example illustrated the differentiating capability (i.e., pluripotency) of the EBs formed by the method of this invention. Pluripotency of the EBs formed according to the procedure described in Example 1.2.3 was confirmed by the detection of the expressed genes (FIG. 3) or proteins (FIG. 4) originated from cells of each of the three germ layers. Total RNA of EBs at 11 days were analyzed for genes expressed in endoderm (such as genes of α-fetoprotein (α-FP) and GATA4), mesoderm (such as genes of cardiac actine, enolase and renin) and ectoderm (such as genes of keratin and neuro filament heavy protein (NFH)) by use of reverse transcription polymerase chain reaction (RT-PCR). Result was illustrated in FIG. 3. Expression of cell genes such as α-FP, GATA4, cardiac actine, enolase and NFH of EBs derived from the HES cells cultured in mECM or hECM (FIG. 3, lanes 3 and 4, respectively) are comparable to those of EBs derived from the HES cells cultured in the presence of PMEF feeder cells (FIG. 3, lane 2, positive control), indicating that EBs formed by this method were pluripotent and were able to differentiate into any cell types of all three germ layers if proper induction was made. This differentiating capability was further confirmed in Example 1.3.2, which will be described with further details in paragraphs below. FIG. 4 illustrated the expression of proteins of corresponding genes such as α-FP of endoderm, actin of mesoderm and nestin of ectoderm for EBs at 30 days.

1.3.2 Differentiation of EBs of Example 1.2.3 into Hepatic-Lineage Cells

EBs can be differentiated into a variety of cell lineages. This example illustrated EBs formed by the method of this invention, particularly, EBs of Example 1.2.3, gave rise to hepatic-lineage cells upon induction with inducing factor. Briefly, after culturing in suspension for 10 days, EBs were harvested and plated onto 24-wells dishes pre-coated with 0.1% laminin. To induce differentiation, 10 ng/ml basic fibroblast growth factor (bFGF) was added to the culture medium after day 14. Cell samples were collected on day 17 for RT-PCR analysis for hepatocyte specific genes

RT-PCR In brief, total RNA was extracted from cells using Rneasy Mini Kit (QIAGENE, Tokyo, Japan). The PCR primer sequences and the length of the amplified products were as follows:

| | |
|---|---|
| | Primer Sequences and the Length |
| Gene | of the Amplified Product |

| | |
|---|---|
| β-actin | 5′-TGGCACCACACCTTCTACAATGAGC-3′ |
| | and |
| | 3′-GCACAGCTTCTCCTTAATGTCACGC-5′; 387 bp |
| Oct-4 | 5′-GACAACAATGAGAACCTTCAGGAGA-3′ |
| | and |
| | 3′-TTCTGGCGCCGGTTACAGAACCA-5′; 217 bp |
| α-FP | 5′-AGAACCTGTCACAAGCTGTG-3′ |
| | and |
| | 3′-GACAGCAAGCTGAGGATGTC-5′; 675 bp |
| ALB | 5′-CCTTTGGCACAATGAAGTGGGTAACC-3′ |
| | and |
| | 3′-CAGCAGTCAGCCATTTCACCATAGG-5′; 350 bp |
| G6P | 5′-CAGGACTGGTTCATCTTGGT-3′ |
| | and |
| | 3′-CAGACATTCAGCTGCACAGC-5′; 421 bp |
| TAT | 5′-CTAGAAGCTAAGGACGTCAT-3′ |
| | and |
| | 3′-GAGGAAGCTCAGAGTGTTGT-5′; 642 bp |
| CYP3A4 | 5′-CAAGACCCCTTTGTGGAAAA-3′ |
| | and |
| | 3′-TGCAGTTTCTGCTGGACATC-5′; 398 bp |

RT-PCR was performed by QIAGENE one step RT-PCR kit in accordance with the following conditions: reverse transcription at 50° C. for 30 minutes; initial PCR activation and denaturation at 95° C. for 15 minutes and 30 seconds; annealing at 52-60° C. for 30 seconds (specifically, 56° C. for α-FP, 68° C. for albumin, 62° C. for glucose-6-phosphatase (G6P) and tyrosin aminotransferase (TAT), and 60° C. for Oct-4 and βP-actin); elongation at 72° C. for 1 minute with total 25 cycles; and final extension at 72° C. for 7 minutes. α-FP was used an indicative of endodermal differentiation as well as an early fetal hepatic marker. Albumin and G6P was used as a marker for liver development at late stage or perinatal stage, and TAT was used an enzymatic maker for perinatal or postnatal hepatic differentiation. Oct-4 and β-actin were used as an undifferentiated ES marker and an endogenous control.

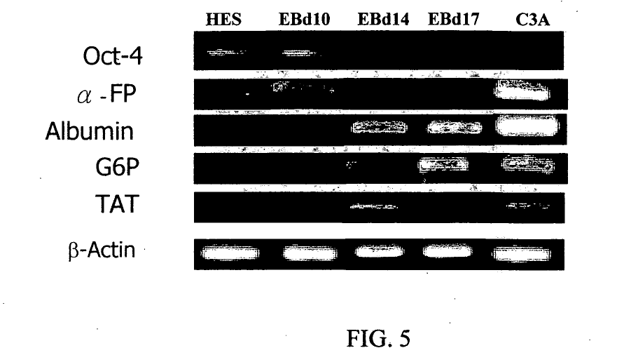

RT-PCR confirmed the expression of the endodermal-specific gene of α-FP decreases as liver develops. In contrast, the expression of three hepatic markers including albumin, G6P and TAT increased upon induction with growth factors when compared with the undifferentiated HES cells (FIG. 5).

### Example 2

### Formation of Hepatic Spheroids

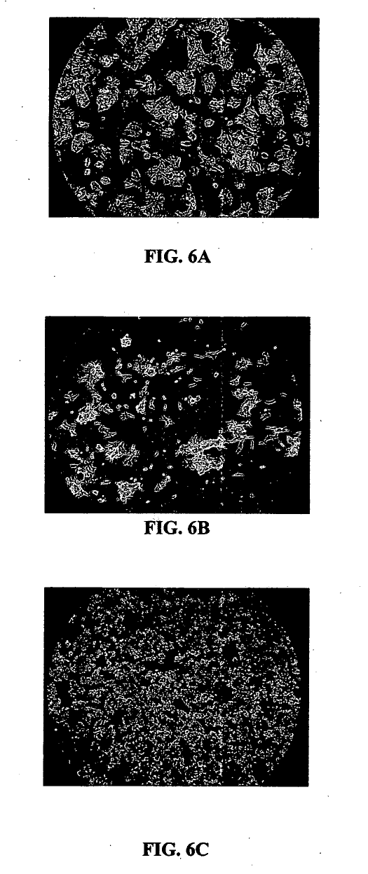

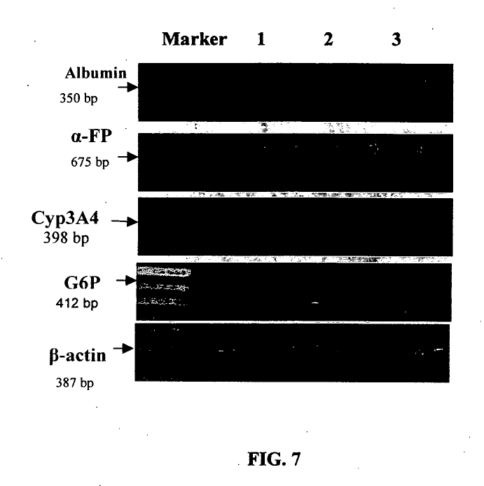

Human hepatoma cell line, i.e., C3A cells, were plated in DMEM medium supplemented with 10% FBS. Cells were maintained at 37° C. and 5% CO₂ and in a water-saturated atmosphere until they reached 90% confluence, then the cells were trypsinized, counted and re-plated onto the cell culture dishs, conventional low attachment Petri dishes (Falcon) or the MC-gel coated culture dishes of Example 1.2.2. FIG. 6A illustrated the morphology of C3A cells attached culture on cell culture dish, FIG. 6B illustrated the partial formation of spheroids for cells maintained in a low attachment Petri dishes for 4 days, most cells showed attached growth on the photograph. In stark contrast, most of the cells maintained in the MC-gel coated culture dish of this invention for 4 days formed spheroids (FIG. 6C), and their expression of hepatic-specific genes such as α-FP, albumin, G6P and CYP 3A4 were illustrated in FIG. 7. (Lane 2)

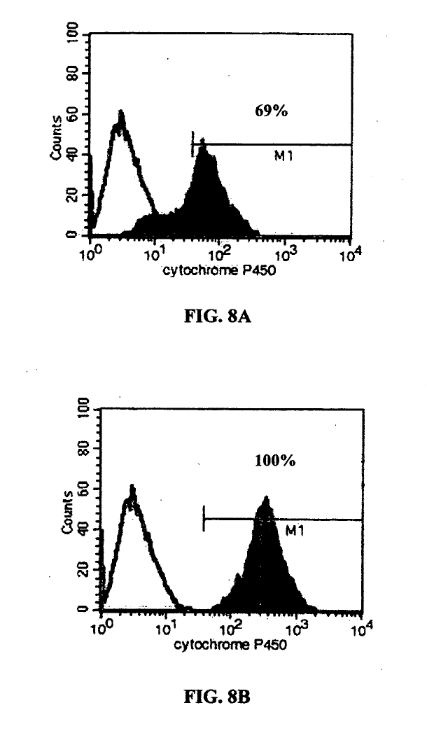

Hepatic Enzyme Activity Measurement Hepatocyte specific enzyme activity, such as the activity of CYP3A4 of C3A cells were measured by pentoxyresorufin (PROD) assay (Molecular Probe) in accordance with the manufacturer's instructions. Briefly, C3A cells grown on different dishes were stained with the fluorescent PROD substrate of CYP at 37° C. for 30 min, which emitted fluorescent light upon catalyzation by CYP. The change in fluorescence intensity was thus used as an indicative of the expression of the hepatic specific enzyme, i.e., CYP, and the fluorescence intensity was determined by use of flow cytometry with excitation wavelength set at 571 nm and emission wavelength set at 585 nm. Result was illustrated in FIG. 8. Compared with the attached cells (FIG. 8A), the suspension spheroids of C3A cells cultured for 7 days (FIG. 8B) expressed high CYP3A4 enzyme activity.

### INDUSTRIAL APPLICABILITY

It is an advantage of the present invention that it provides a method of forming spheroids or embryoid bodies, which is easy to use and cost-effective, with multicellular spheroid or embryoid body formation ratio over 90%, far superior to that of the conventional hanging drop techniques and/or the low attachment dishes. Another advantage of the present invention is to provide an easy means of forming embryoid bodies suitable for differentiation into other cell types, such as hepatocytes and chondrocytes. Still another advantage of this invention is to provide spheroids of functional cells including cell lines, primary cells, stem cell derived cells for various clinical applications such as transplantation, drug screening in an effective and cost-economic manner.

The foregoing description of various embodiments of the invention has been presented for purpose of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method of forming multicellular spheroids, comprising culturing spheroid-forming cells in a culture vessel pre-coated with cellulose and/or its derivatives.

2. The method of claim 1, wherein the spheroid-forming cells include stem cells and tissue specific cells.

3. The method of claim 2, wherein the stem cells include pluripotent stem cells, adult stem cells and stem cells derived cells.

4. (canceled)

5. The method of claim 1, wherein the cellulose is selected from the group consisted of methylcellulose (MC), carboxymethylcellulose (CMC), hydroxymethylcellulose (HMC), hydroxyethylcellulose (HEC), hydroxypropylcellulose (HPC) and hydroxypropylmethylcellulose (HPMC).

6. A method of forming embryoid bodies, comprising culturing undifferentiated embryonic stem cells in a culture vessel pre-coated with cellulose and/or its derivatives.

7. The method of claim 6, wherein the cellulose is selected from the group consisted of methylcellulose (MC), carboxymethylcellulose (CMC), hydroxymethylcellulose (HMC), hydroxyethylcellulose (HEC), hydroxypropylcellulose (HPC) and hydroxypropylmethylcellulose (HPMC).

8. The method of claim 6, wherein at least 90% of undifferentiated stem cells form embryoid bodies.

9. The method of claim 8, wherein the embryoid bodies will differentiate into mature cell types.

10. The method of claim 8, wherein the mature cell types include cells with hepatic function.

11. A method of forming hepatic spheroids, comprising culturing cells with hepatic function in a culture vessel pre-coated with cellulose and/or its derivatives.

12. The method of claim 11, wherein the cellulose is selected from the group consisted of methylcellulose (MC), carboxymethylcellulose (CMC), hydroxymethylcellulose (HMC), hydroxyethylcellulose (HEC), hydroxypropylcellulose (HPC) and hydroxypropylmethylcellulose (HPMC).

13. The method of claim 11, wherein the cells with hepatic function are human cells.

14. The method of claim 6, wherein the undifferentiated embryonic stem cells are undifferentiated human embryonic stem cells.

15. The method of claim 1, wherein the spheroid-forming cells are human spheroid-forming cells.
